(19) United States
(12) Patent Application Publication
(10) Pub. No.: US 2012/0104262 A1
(43) Pub. Date: May 3, 2012

(54) EVALUATION OF MEASUREMENTS FROM A PIXELATED DETECTOR

(75) Inventors: Jens WIEGERT, Aachen (DE); Klaus Juergen ENGEL, Aachen (DE)

(73) Assignee: KONINKLIJKE PHILIPS ELECTRONICS N.V., EINDHOVEN (NL)

(21) Appl. No.: 12/631,878

(22) Filed: Dec. 7, 2009

(30) Foreign Application Priority Data
Dec. 8, 2008 (EP) 08170956.0

(51) Int. Cl. G01T 1/166; G01T 1/164; G01T 1/00; A61B 6/03; G01N 23/04

(52) U.S. Cl. 250/363.03; 378/19; 378/62; 250/395; 250/363.04; 250/336.1

(57) ABSTRACT

The invention relates to a method and a data processing device for evaluating measurement signals provided by a layered, pixelated radiation detector. A generalized detector response function is provided that describes the energy-related crosstalk caused by radiation incident in the d-th neighboring pixel. With the help of this GDR-function, crosstalk effects can be taken into account to achieve a more accurate determination of imaging parameters related to an imaged object. The approach can particularly be used in spectrally resolved, photon counting CT detectors with small, layered pixels.

### FIELD OF THE INVENTION

The invention relates to a method and a data processing device for evaluating measurement signals from a pixelated radiation detector. Moreover, it relates to an imaging system comprising such a device and to a computer program product, a data carrier, and a transmission method related to the method.

### BACKGROUND OF THE INVENTION

The U.S. Pat. No. 7,208,739 B1 discloses a radiation detector comprising a plurality of pixels in which incident radiation is converted into electrical charges. The document further discloses a method to correct measurement signals for a pile-up of signals and for a sharing of charges between adjacent pixels.

### SUMMARY OF THE INVENTION

Based on this situation it was an object of the present invention to provide means for improving the evaluation of measurement signals from a radiation detector with respect to an object imaged by said detector.

This object is achieved by a data processing device according to claim 1, a method according to claim 2, an imaging system according to claim 13, a computer program product according to claim 14, and a data carrier according to claim 15. Preferred embodiments are disclosed in the dependent claims.

According to its first aspect, the invention relates to a data processing device for evaluating measurement signals that are provided by a radiation detector, said radiation detector having a plurality of N>1 pixels with each pixel having a number of L≧1 layers. The pixels will in the following be numbered with the variable p (1≦p≦N), and the layers with the variable l (1≦l≦L). As usual, a “pixel” is a detector element providing a signal that relates to one point (“picture element”) of the image of an object that is generated with the detector. In the present case, the pixels may optionally be layered (if L>1), i.e. consists of several sub-units arranged in different layers one behind the other in the direction of radiation incidence, each sub-unit providing a measurement signal of its own that is related to the same pixel-position in the generated image. Moreover, the pixels are typically arranged in a one- or two-dimensional array. The data processing device may be realized by dedicated electronic hardware, digital data processing hardware with associated software, or a mixture of both. It comprises the following components:

a) A “crosstalk module” for providing a function ƒ^{(l,d)} (Eᵢₙ, Eₒᵤₜ), which will be called “generalized detector response function” or a shortly “GDR-function” in the following. The GDR-function describes the contribution of photons, which hit the first layer of a given pixel p and have an impinging energy Eᵢₙ, to a measurement component at a deposited energy Eₒᵤₜ in the l-th layer of the d-th neighbor pixel. In this context, the integer variable d ranges between 0 and a given positive number dₘₐₓ and may be just an arbitrary numbering of neighbor pixels. Preferably, the variable d relates however to an ordering of pixels with respect to their distance from the considered central pixel p, the nearest neighbors corresponding for example to d=1, the next but one nearest neighbors to d=2 etc. It should be noted that, if the detector configuration is not isotropic with respect to the pixel position, the GDR-function will additionally depend on the considered central pixel p (e.g. expressed as ƒ^{(p,l,d)} (Eᵢₙ, Eₒᵤₜ)).

The crosstalk module may comprise a memory in which parameters of a numerical representation of the GDR-function are stored, for example as a lookup table. Moreover, the GDR-function may be provided explicitly or implicitly (e.g. via a function or relation that is equivalent to or a derivative of the GDR-function).

b) An “evaluation module” for determining radiation related parameters of an object, wherein radiation from said object reaches the detector, and wherein said determination is based on measurement signals and on the GDR-function.

The invention further relates to a method for evaluating measurement signals from a radiation detector with a plurality of N>1 pixels having a number of L≧1 layers, comprising:

a) Providing (explicitly or implicitly) a GDR-function which describes the contribution of radiation of energy Eᵢₙ incident on the first layer of a pixel to a measurement component at deposited energy Eₒᵤₜ in the l-th layer (1≦l≦L) of the d-th neighbor pixel (0≦d≦dₘₐₓ, dₘₐₓ>0).
b) Determining parameters of an object from which radiation reaches the detector, wherein said determination is based on measurement signals and the GDR-function.

The described data processing device and the associated method allow the image-related evaluation of measurement signals from a radiation detector with improved accuracy as they take into account crosstalk effects between different pixels and optionally also between different layers of the detector in a spectrally resolved way. This possibility is particularly advantageous in spectral X-ray detectors using photon counting, which are typically subdivided into small, layered pixels to limit the counting rate each pixel layer has to deal with. The small pixel size causes an increase of crosstalk between pixels, for example due to Compton scattering or K-edge fluorescence, particularly if the detector contains materials with a low atomic number Z (e.g. silicon, Si). This crosstalk is efficiently compensated for by the described method.

In principle, the GDR-function that is provided by the crosstalk module may be analytically derived from theoretical considerations. As the underlying mechanisms are however complicated, the GDR-function may preferably be determined from simulations of the radiation detector, for example with a Monte Carlo procedure. Moreover, the GDR-function may (partly or completely) be determined experimentally, for example by irradiating a single pixel with monochromatic radiation and by measuring the resulting signals from the other pixels and layers. The GDR-function may be expressed after determination for example by a lookup table or by parameters of a fitted parametric analytical expression.

The measurement signals that are provided by the radiation detector may in general correspond to any value that is related to the incident radiation. Thus they may for example represent the total number of photons of the incident radiation that hit a considered pixel and layer during a given time interval, or the total energy of these photons. Preferably, the measurement signals provide energy resolved (i.e. spectral) information about the photons of the incident radiation, for example if they represent the amount of radiation that was detected in the l-th layer of a considered pixel with respect to a plurality of given energy windows (or “bins”). Said amount of radiation may for example be expressed by the total energy of all detected photons in said energy window, or preferably by the number of said photons. Measurement signals of this kind can for instance be obtained by evaluating electrical pulses generated in a direct conversion material by incident photons with respect to their shape (height) and number.

The object parameters that are determined by the evaluation module can in general comprise any value that is related to the interaction of the object with the radiation measured by the detector. In a typical scenario, the radiation detector is used to generate a transmission image of an object, i.e. the object is irradiated (e.g. by an X-ray source) and the amount of radiation that passes the object is measured by the radiation detector as a projection image. The object parameter that is determined in this case is the attenuation coefficient describing the local absorption of incident radiation inside the object. Preferably, said attenuation coefficient is split into a number of J≧1 components that are related to different physical effects, for example to the photo effect, to Compton scattering, and/or to K-edge absorption of a particular material. Separate determination of these different components of the attenuation coefficient provides additional information, which is for example valuable in clinical X-ray examinations of patients.

In a further development of the aforementioned approach, the object parameters that are related to the J≧1 components of the attenuation coefficient comprise integrals of said components in regions of the object that are “in front of” a given pixel (i.e. radiation that hits the considered pixel is transmitted through said regions). The consideration of integrals along ray paths takes the fact into account that only such integral values can be determined in transmission measurements. As is well known to a person skilled in the art, the spatial distribution of the attenuation coefficient or of its components inside an object can however be calculated in a “Computed Tomography” (CT) procedure from a plurality of such integrals which are determined for different directions of radiation.

In general, the object parameters that shall be determined are associated via some relation to the measurement signals provided by the radiation detector. Conversion of this relation allows to calculate the object parameters of interest from said measurement signals either exactly (e.g. if there are as many unknown object parameters as measurement signals) or approximately (e.g. if there are less or more measurement signals than object parameters). A preferred approach to determine the object parameters is based on the optimization of a Maximum Likelihood function which describes the measurement results as one realization of a stochastic process that depends on the object parameters. A set of object parameters can then be determined that yields the highest probability for the observed measurement signals.

The Maximum Likelihood function may particularly be based on a modeled Poisson distribution of radiation detection events taking place in the l-th layer of a pixel p. The Poisson distribution is usually an appropriate model for a statistically independent behavior of photons.

The determination of the object parameters may be done in one step, for example if a single analytical solution is possible. In other cases, the determination may preferably be done iteratively in several steps, wherein in each step only a part of mutually dependent object parameters is adapted.

For example, the measurement signal provided by some layer of a given pixel p is typically caused primarily by radiation that hits this pixel directly, said radiation depending on first object parameters (e.g. the line integrals of attenuation components along beam paths directed to the considered pixel p). Due to crosstalk effects, the measurement signal will to some extent also depend on the amount of radiation hitting neighboring pixels, which radiation depends on second object parameters (e.g. line integrals of attenuation components along beam paths directed to the neighboring pixels). For the purpose of the iteration, only the first object parameters related to the considered pixel p may be adjusted (e.g. via a Maximum Likelihood optimization) in a given iteration step while the second object parameters corresponding to neighboring pixels are kept constant (i.e. taken from previous iteration steps). In this way the computational effort can be limited, allowing for example a parallelization of the corresponding calculations.

According to a preferred embodiment of the iterative process, cross-talk corrected object parameters are determined in an iteration step n+1 based on the measurement data and the distribution of impinging energy (i.e. energy incident on the first layer of the pixels) that was derived in a previous iteration step n from the previous object parameters and the GDR-function.

In another embodiment of the iterative determination of object parameters, said determination starts with an approximation that takes no crosstalk between neighboring pixels into account. This approximation corresponds to the typical state of the art in the evaluation of measurement data from a radiation detector and therefore already provides a solution that lies close to the exact results.

The invention further relates to an imaging system comprising a radiation detector and a data processing device of the kind described above. The imaging system may particularly be an X-ray, CT (Computed Tomography), PET (Positron Emission Tomography), SPECT (Single Photon Emission Computed Tomography) or nuclear imaging system.

The method according to the invention will typically be realized with the help of a computing device, e.g. a microprocessor or an FPGA. Accordingly, the present invention further includes a computer program product which provides the functionality of any of the methods according to the present invention when executed on a computing device.

Further, the present invention includes a data carrier, for example a floppy disk, a hard disk, an EPROM, or a compact disc (CD-ROM), which stores the computer product in a machine readable form and which executes at least one of the methods of the invention when the program stored on the data carrier is executed on a computing device. The data carrier may particularly be suited for storing the program of the computing device mentioned in the previous paragraph.

Nowadays, such software is often offered on the Internet or a company Intranet for download, hence the present invention also includes transmitting the computer product according to the present invention over a local or wide area network.

### BRIEF DESCRIPTION OF THE DRAWINGS

These and other aspects of the invention will be apparent from and elucidated with reference to the embodiment(s) described hereinafter. These embodiments will be described by way of example with the help of the accompanying drawings in which:

FIG. 1 schematically shows a scenario in which a data processing device according to the present invention is used;

FIGS. 2 and 3 represent formulae related to the evaluation procedure executed by the data processing device;

FIG. 4 shows a section through a particular radiation detector with intermediate Si layers;

FIG. 5 shows crosstalk signals generated in neighboring pixels in the detector of FIG. 4;

FIG. 6 shows a simulation model that was imaged with a procedure according to the present invention;

FIGS. 7-9 show measurement results obtained with the simulation model of FIG. 6.

Like reference numbers in the Figures refer to identical or similar components.

### DESCRIPTION OF PREFERRED EMBODIMENTS

Spectral CT based on pulse counting in a direct-converting (DiCo) detection material aims at measuring the energy-spectrum of X-ray photons having passed an object to be scanned. The associated problem of the extremely high count rates behind object areas close to the direct beam as well as in the direct beam can be solved by going to a sub-pixelation (with a typical pixel pitch of about 300 μm), by structuring the DiCo-based detector in several layers (with a typical thicknesses of 500 μm and more), and by accepting that “saturated” top layers (i.e. the count rate exceeds a given maximum, so that photons can no longer be correctly separated) do not provide a measurement result, as long as there are bottom layers, which still do.

However, due to several crosstalk mechanisms (e.g. Compton scatter, K-fluorescence, charge sharing), any pixel sees a signal component caused by its neighboring pixels or even from pixels in the layers above or below the considered pixel. Especially if the detector also contains low-Z materials like Si, the amount of crosstalk due to Compton scatter or K-fluorescence may become considerable, which has an adverse effect on the image quality of the reconstructed Spectral CT images and in particular on the quantitative results of a K-edge material mass density in a scanned body, for example the density of Gd or Iodine which are used as contrast agents e.g. in cardiac imaging.

To address the aforementioned issues, it is proposed here to describe inter-pixel crosstalk effects by means of a generalized detector response (GDR) function, which incorporates the response of a considered pixel to the illumination of neighboring pixels (in the same layer or in different layers). This generalized detector response can be efficiently used in a Maximum-Likelihood approach to find the coefficients of the decomposition of the attenuation coefficient modeling a scanned object (the latter procedure is for example described in: Roessl E. and Proksa R., “K-edge imaging in x-ray computed tomography using multi-bin photon counting detectors”, Phys. Med. Biol. 52, 4679-4696).

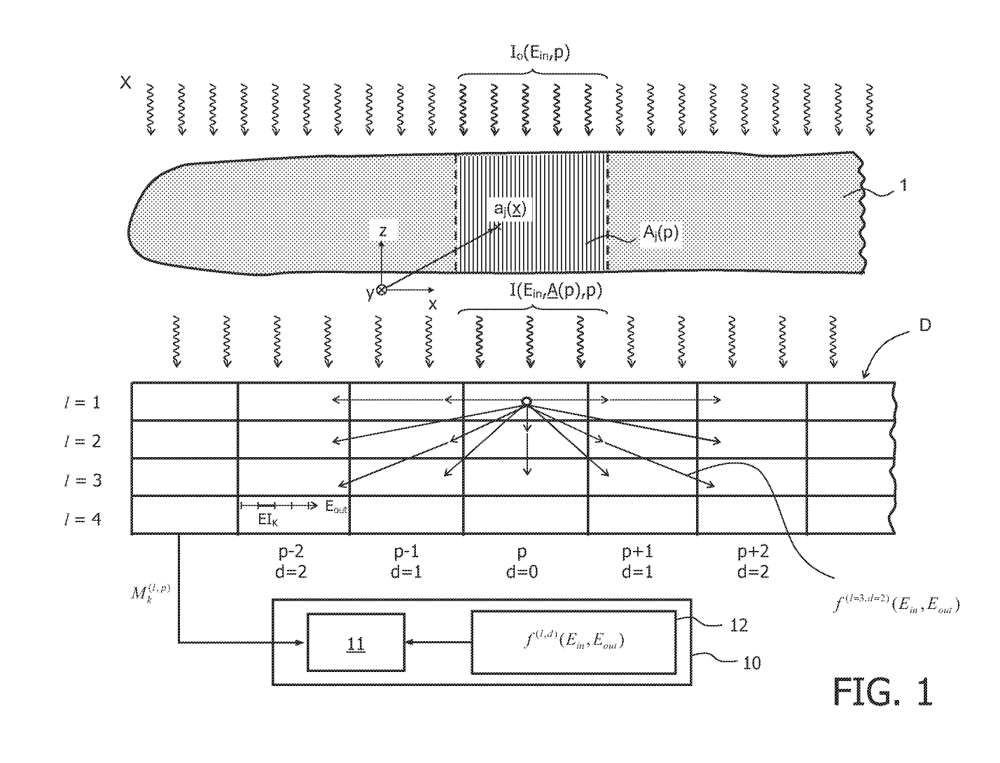

In the following, the above general concepts will be described in more detail with respect to a particular embodiment that is illustrated in FIG. 1. The top of said Figure shows X-radiation X coming from some radiation source (not shown) with a given intensity and spectral composition. Considering only those rays that are directed to a given pixel p of the detector D, the function I₀(Eᵢₙ,p) describes the amount of said radiation that has an energy Eᵢₙ, wherein this energy Eᵢₙ ranges between some lower limit Eₘᵢₙ and upper limit Eₘₐₓ according to the characteristics of the X-ray source.

The considered X-radiation next passes through an object 1 that shall be imaged, for example the body of a patient. When passing through said object 1, the radiation is attenuated according to the distribution of the spatial components aⱼ(x) of the attenuation coefficient μ (E,x) (with 1≦j≦J).

Behind the object 1, the considered radiation propagates towards pixel p of the detector D with a reduced amount I(Eᵢₙ, A(p), p). This amount depends on the integrated values A(p)=(A₁(p), A₂(p), . . . A_{J}(p)) of the attenuation coefficient components aⱼ(x) in the region of the object that is passed by the X-rays directed to pixel p (usually this region can be approximated by a line as the lateral pixel size is small in relation to the object thickness).

The detector D is structured in a plurality of N≧2 pixels numbered p, p±1, p±2, . . . , each pixel comprising a number of L≧1 layers. In the shown example, L=4 layers with numbers l=1, l=4 are present, the signals of which can separately be read out. It should be noted that the Figure shows only a few pixels of a usually much larger number, and that these pixels typically have a two-dimensional arrangement in the x- and y-direction of the shown coordinate system.

The detector D is connected to a data processing device 10 that reads out and processes the measurement signals Mₖ^{(l,p)} provided by the detector. Each of these measurement signals Mₖ^{(l,p)} represents the number of photons counted in the layer l of pixel p that have an energy Eₒᵤₜ in an energy window EIₖ, wherein the observed energies are subdivided into K≧1 given energy windows or bins EI₁, . . . EI_{K}. The measurement signals Mₖ^{(l,p)} are processed by an evaluation module 11 to determine characteristic parameters of the object 1, particularly the integrals A(p).

As mentioned above, an X-ray photon that hits the first layer l=1 of the pixel p will usually entail crosstalk signals in all other layers of said pixel and in all layers of the neighboring pixels p±1, p±2, . . . , which contribute to the measurement signals of these layers. To take this into account, the generalized detector response or GDR-function ƒ^{(l,d)} (Eᵢₙ, Eₒᵤₜ) is used, which is stored in an appropriate form in a crosstalk module 12 of the data processing device 10.

The values ƒ^{(l,d)} (Eᵢₙ, Eₒᵤₜ) of the GDR-function describe in relative frequencies, the fraction of quanta of energy Eₒᵤₜ of a large number of X-ray photons of energy Eᵢₙ, which enter the very first layer of a considered illuminated layered pixel p, that are deposited in the l-th layer of the d-th neighbor pixel (with 1≦l≦L and 0≦d≦dₘₐₓ, where d=0 refers to the actually illuminated (layered) pixel p). In practice, this GDR-function can be determined by theoretical considerations (e.g. Monte Carlo simulations of a real direct converting sensor), as assumed in the following, or by dedicated experiments, with a large number of photons incident on a particular pixel of a real detector.

Usually the generalized detector response function refers to a two-dimensional detector, one dimension being in parallel to the axis of rotation of the detector in CT imaging (y-axis in FIG. 1), which rotates around the patient 1, and the other dimension being in parallel to the direction of rotation (x-axis in FIG. 1; often also called φ-direction (phi)); hence each illuminated pixel has (2d+1)²−(2(d−1)+1)²=8d neighbor pixels in distance d, which are located on a square margin in distance d around the considered illuminated pixel.

In the following an iterative procedure will be described by which the object parameters A(p) can be determined.

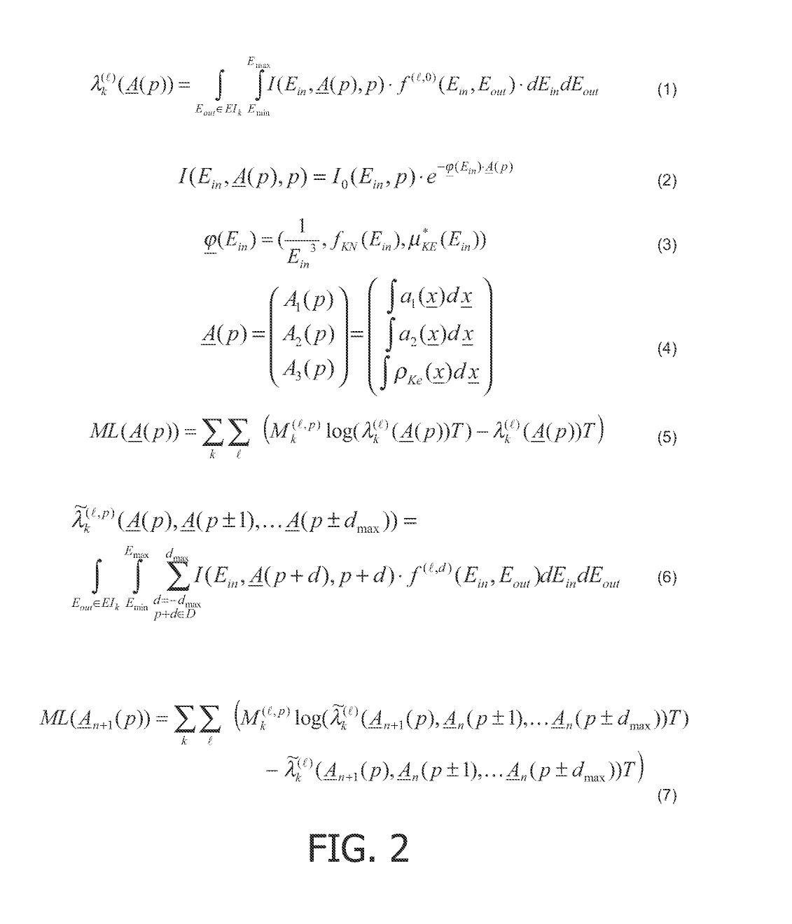

The first iteration step of this procedure tries to determine good starting values for the object parameters A(p) by first neglecting inter-pixel crosstalk. The search for the decomposition coefficients aⱼ(x) can then be done by modeling the mean value of the Poisson processes describing the arrival rates λₖ^{(l)} (A(p)) of the k-th energy window EIₖ in the l-th layer in pixel p (cf. Roessl E. and Proksa R., above). The corresponding formulae are given by equations (1) to (4) of FIG. 2, in which:
the integrals in equation (4) are taken along the X-ray paths through the object 1;
ƒ_{KN}(E) represents the Klein-Nishina formula for representing the energy dependency of the Compton effect;
μ_{Ke}*(E) is the energy dependence of the mass attenuation coefficient of a material with K-edge, which is used as contrast agent, e.g. Gd;
ρ_{Ke}(x) is the mass density of the aforementioned material.

To find the desired object parameters A(p) for each pixel p, a possible approach is to maximize for each pixel independently the Maximum Likelihood function of equation (5), where Mₖ^{(l,p)} represents the count value in the k-th energy window of the l-th layer of pixel p, and T is the measurement time. This allows for a high parallelization of the maximization process, i.e. in principle the solution can be searched for each pixel in a separate computing entity.

In a second iteration step, the solutions A(p) for all N pixels, which were found in the first iteration step, are used as a starting value to solve a similar problem in which inter-pixel crosstalk is taken into account. The corresponding formula is given in equation (6) of FIG. 2. This formula refers to a one-dimensional detector like the one shown in FIG. 1, and the condition “p+dεD” shall denote that only those pixels are comprised by the sum that are actually still within the detector D.

With the modified arrival rates \tilde{λ}ₖ^{(l,p)}(A(p), A(p±1), . . . A(p±dₘₐₓ)) of the k-th energy window in the l-th layer in pixel p (of all N pixels), where the modification takes into account the inter-pixel crosstalk contribution to the arrival rate actually seen by the considered pixel, one maximizes the same Maximum Likelihood function of equation (7). While this maximization could in principle be done simultaneously with respect to all object parameters A(p), A(p±1), . . . A(p±dₘₐₓ), it is preferred that the maximization is done only for a single object parameter at a time, which is written in equation (7) as the variable Aₙ₊₁(p) of the (n+1)-th iteration step and of the pixel p under consideration, while the residual parameters A(p±d) for d≧1 are considered as constant and represented by the values Aₙ(p±d) from the previous iteration step n. In this case, the maximization is again an easy task to parallelize.

After the optimal value Aₙ₊₁(p) has (at least approximately) been determined, similar procedures are executed for the residual pixels p′≠p to determine also the (n+1)-th iteration value of their object parameters, i.e. the Aₙ₊₁(p′). When this has been done for all pixels, the next iteration stage (n+2) can begin with e.g. again pixel p. The iteration will typically be ended when the results approach stationary values.

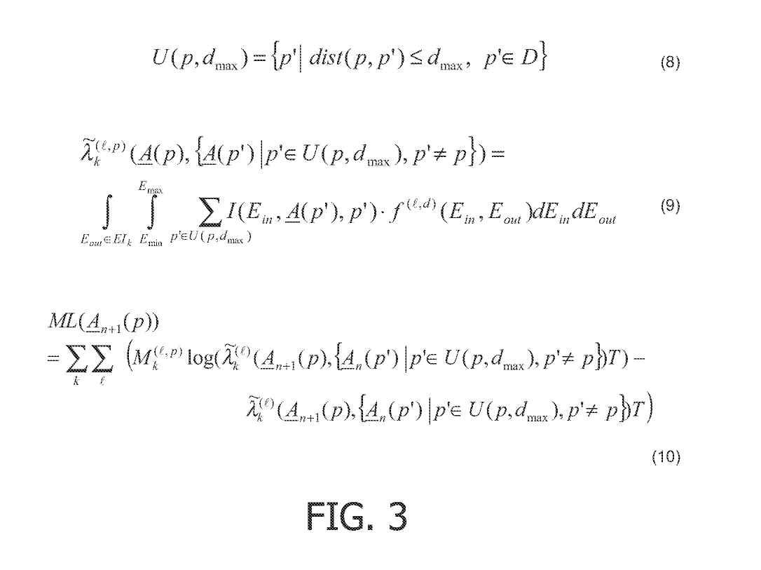

The formulae of FIG. 3 refer to a generalization of equations (6) and (7) that comprises also the case of a two-dimensional detector D. To this end a neighborhood U(p,dₘₐₓ) is defined in equation (8) with the help of some distance measure dist(p,p′) between pixels p and p′. This may for example be a measure that assigns the value “1” to the nearest neighbors p′ of a pixel p, the value “2” to the next but one nearest neighbors etc. (of course dist(p,p) should be zero). Equations (9) and (10) are then the direct equivalents of equations (6) and (7), respectively.

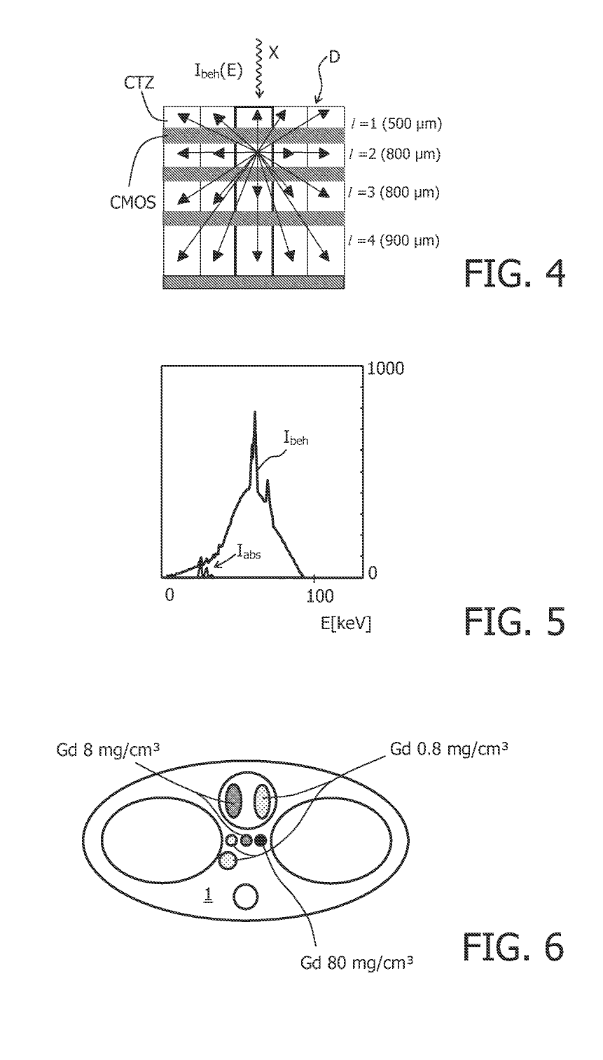

In the following, several simulation results will be presented. First, FIG. 4 shows a multi-layer detector D, here as an example with four CZT layers l=1 to l=4. The “CMOS” layers represent the readout electronics implemented on a CMOS integrated circuit (modeled by a pure Si layer).

FIG. 5 shows the spectrum I_{abs} of absorbed energy E for the next neighbor pixels in the top layer l=1 of the detector D of FIG. 4 (resulting from the simulated detector response modeling the X-ray interaction processes only) as obtained for a given spectrum I_{beh}(E) behind an object.

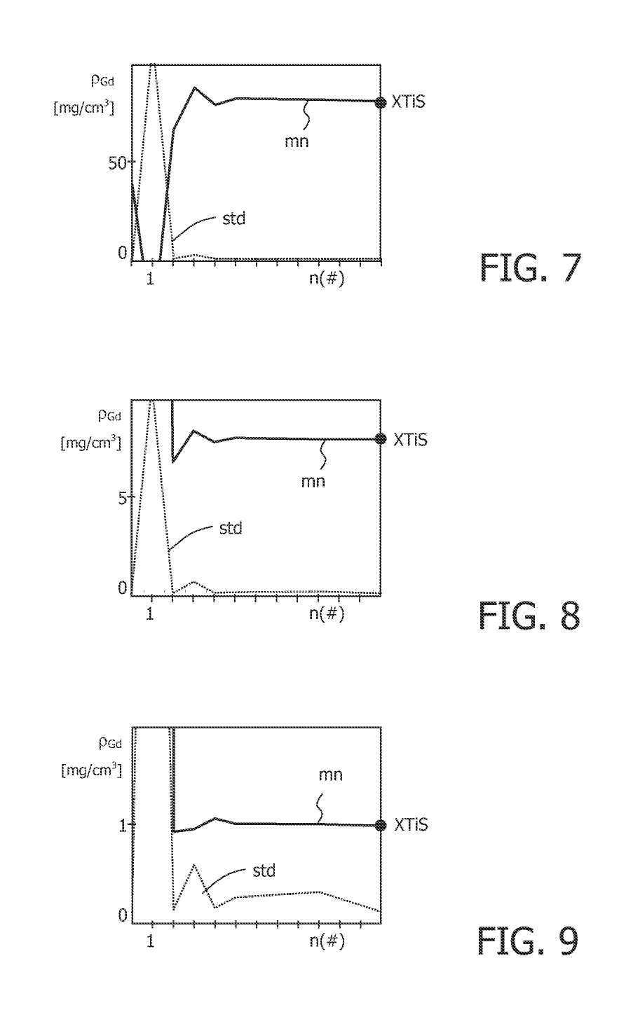

Results of the described data processing approach are shown in FIGS. 7 to 9. FIG. 7 displays the reconstructed Gd mass density ρ_{Gd} measured for the small right-hand side vessel of the test object 1 shown in FIG. 6 as a function of the number n of crosstalk iterations (with “mn” corresponding to the mean value and “std” to the standard deviation). The detector consisted of eight Si layers with thicknesses 10 mm, 10 mm, 8 mm, 8 mm, 6 mm, 6 mm, 5 mm, 4.6 mm, and a pixel size of approximately (1.5 mm)² The measurement result improves considerably with the number of correction iterations, wherein the “ideal” result is represented by the dots “XTiS” on the right hand side (which means ideal suppression of crosstalk e.g. by suitable absorbing separators between pixels).

FIGS. 8 and 9 show the reconstructed K-edge material mass density ρ_{Gd} in the left ventricle and the right ventricle, respectively, of the test object 1 shown in FIG. 6. The data were measured with the same (pure Si based) detector as in FIG. 7.

It turns out that especially in case of Spectral CT detector concepts based partly (e.g. 5 Si layers of 1 mm thickness, and 3 layers of CZT of 1 mm thickness below the Si layers) or fully (only using several Si layers) on lower-Z materials (like Si), elimination of crosstalk by processing with the described GDR-function greatly improves the resulting measurement values such as the mass density of a K-edge material (e.g. Gd) contained in the scanned object.

In addition, it was found that the technique is also very valuable, if the detector layers are based on high-Z material, however the limitation of the acceptable count rate within a detector layer (until the layer is considered “saturated”, e.g. since the material cannot distinguish between successive photons, since their rate is too high) is very conservative e.g. 1 Mcps.

The described technique can also be applied to “hybrid” detectors, e.g. a detector with L-1 energy-resolving layers (such as Si or CZT) and a last (L-th) layer that only integrates (e.g. GOS).

A main application of the invention is Computed Tomography with energy resolution, projection imaging with energy resolution, or any other application that may benefit from energy-resolving X-ray photon counting.

Finally it is pointed out that in the present application the term “comprising” does not exclude other elements or steps, that “a” or “an” does not exclude a plurality, and that a single processor or other unit may fulfill the functions of several means. The invention resides in each and every novel characteristic feature and each and every combination of characteristic features. Moreover, reference signs in the claims shall not be construed as limiting their scope.

1. A data processing device for evaluating measurement signals from a radiation detector with a plurality of N>1 pixels having a number of L≧1 layers, comprising:
a) a “crosstalk module” for providing a generalized detector response function, called GDR-function, which describes the contribution of radiation of energy incident on the first layer of a pixel to a measurement component at deposited energy in the l-th layer of the d-th neighbor pixel;
b) an evaluation module for determining parameters of an object from which radiation reaches the detector, wherein said determination is based on measurement signals and the GDR-function.

2. A method for evaluating measurement signals from a radiation detector with a plurality of N>1 pixels having a number of L≧1 layers, comprising:
a) a GDR-function which describes the contribution of radiation of energy incident on the first layer of a pixel to a measurement component at deposited energy in the l-th layer of the d-th neighbor pixel;
b) determining parameters of an object from which radiation reaches the detector, wherein said determination is based on measurement signals and the GDR-function.

3. The method of claim 2,
wherein the GDR-function is derived experimentally or from simulations of the radiation detector.

4. The method of claim 2,
wherein the measurement signals represent the amount of radiation, which was measured in a layer of a pixel, with respect to a plurality of given energy windows.

5. The method of claim 2,
wherein the determined object parameters are related to a given number of J≧1 components of the attenuation coefficient in the object.

6. The method of claim 5,
wherein the object parameters comprise the integrals of said components in regions of the object that are irradiated in front of a pixel.

7. The method of claim 2,
wherein the object parameters are determined from the optimization of a Maximum Likelihood function.

8. The method of claim 7,
wherein the Maximum Likelihood function is based on a modeled Poisson distribution of radiation detection events in the layers of a pixel.

9. The method of claim 2,
wherein the determination is done iteratively, adapting in each step only a part of mutually dependent object parameters.

10. The method of claim 9,
wherein cross-talk corrected object parameters are determined in an iteration step based on the distribution of impinging energy incident on the first layer of the pixels that was derived in a previous iteration step.

11. The method of claim 9,
wherein only the object parameters related to a single pixel are adapted in each iteration step.

12. The method of claim 9,
wherein the determination starts with an approximation that takes no crosstalk between neighboring pixels into account.

13. An imaging system, particularly an X-ray, CT, PET, SPECT or nuclear imaging system, comprising a radiation detector and a data processing device according to claim 1.

14. A computer program product for enabling carrying out a method according to claim 2.

15. A record carrier on which a computer program according to claim 14 is stored.
